(19) United States
(12) Patent Application Publication
(10) Pub. No.: US 2011/0052594 A1
(43) Pub. Date: Mar. 3, 2011

(54) AGENTS THAT DISSOLVE ARTERIAL THROMBI

(75) Inventors: Simon Karpatkin, New York, NY (US); Michael Nardi, New York, NY (US)

(73) Assignee: NEW YORK UNIVERSITY, New York, NY (US)

(21) Appl. No.: 12/369,527

(22) Filed: Feb. 11, 2009

Related U.S. Application Data
(62) Division of application No. 11/472,394, filed on Jun. 22, 2006, now abandoned.
Application No. 11/472,394 is a continuation-in-part of application No. 10/473,034, filed on Jul. 20, 2004, now abandoned.
(60) Provisional application No. 60/278,425, filed on Mar. 26, 2001.

(51) Int. Cl. A61K 39/395; A61P 9/00

(52) U.S. Cl. 424/145.1; 424/158.1

(57) ABSTRACT

Agents that induce platelet fragmentation include an IgG antibody that reacts with platelet epitope GPIIIA49-66 on platelet membrane, recombinant AMANTS-18, phorbol 12-myristate 13-acetate (PMA) and A23817.

### CROSS-REFERENCE TO RELATED APPLICATIONS

The present application is a divisional of U.S. patent application Ser. No. 11/472,394, filed Jun. 22, 2006, which is a continuation-in-part of co-pending parent application Ser. No. 10/473,034 Filed on Jul. 20, 2004, which application was the national stage under 35 U.S.C. 371 of PCT/US02/09249, filed Mar. 26, 2002, and claiming priority from U.S. Provisional Application No. 60/278,425, filed Mar. 26, 2001. The entire contents of these applications are hereby incorporated by reference in their entirety.

### FIELD OF THE INVENTION

The present invention relates to agents that induce platelet fragmentation that can be used to dissolve arterial thrombi.

### BACKGROUND OF THE INVENTION

Thrombus formation is characterized by rapid conformational changes to blood platelets and activation of various plasma proproteins. In response to a range of triggering stimuli and cascading events, zymogenic prothrombin is catalyzed to thrombin. In turn, thrombin acts upon the soluble structure protein fibrinogen, cleaving the N-terminal A and B polypeptides from the alpha and beta chains to form fibrin monomer. Cleavage results in redistribution of charge density and exposure of two polymerization sites, enabling growth of the monomer into an insoluble, three dimensional polymeric network. Concurrently, thrombin acts to induce significant physiological change to a “resting” or inactive blood platelet by changing its shape. This is associated with thromboxane A₂ synthesis and release of ADP from intraplatelet storage granules which enhances platelet aggregation. Such activated platelets play a dual role in hemostasis:

i) They are more adhesive and capable of binding fibrinogen and von Willebrand factor. Activated platelets adhere to subendothelial von Willebrand factor via the GPIb receptor and co-aggregate with fibrinogen and von Willebrand factor via the GPIIbIIIa

ii) Activated platelets act as a catalytic surface for thrombin generation from its plasma pro-enzymes. This results in the formation of insoluble fibrin intermeshed within and around the platelet thrombus. This three dimensional platelet plug under pathophysiological conditions can serve to compromise circulatory system patency leading to tissue infarction and necrosis.

Thrombus formation in the absence of vessel trauma or rupture is pathogenic, and is a causative factor in ischemic heart disease (myocardial infarction, unstable angina), ischemic stroke, deep vein thrombosis, pulmonary embolism, and related conditions.

Appearance of atherosclerotic plaques within the coronary arteries is the precursor to ischemic heart disease (IHD). Disruption of the endothelial layer of coronary arteries by lipid-filled foam cells is followed by microlesions in or rupture of the endothelial wall. Either event results in exposure of platelet activation molecules within the intima, including tissue factor plasminogen activator and collagen. Platelet aggregation results in thrombus formation at the site of plaque rupture. Mural thrombi extend within this ruptured plaque into the vessel volume. Small, non-occlusive mural thrombi may oscillate in response to pressure variations within the vessel, resulting in transient stenosis of the affected channel. Such time-variant blockage is characteristic of unstable angina. Larger, occlusive mural thrombi may completely block the affected vessel, resulting in myocardial infarction and/or patient death.

Causative factors for ischemic stroke include cardiogenic emboli, atherosclerotic emboli, and penetrating artery disease. Cardiogenic emboli are generated within the left atrium and ventricle as a result of valve disease or cardiomyopathy. Migration of the embolus through the aorta into the carotids results in closure of a cerebral vessel. As in Ishemic Heart Disease (IHD), atherosclerotic plaques within the carotids or cerebral vasculature serve as loci for the formation of mural thrombi. Vascular disease can result in hypercoagulable states, resulting in thrombus formation. Consequences of ischemic stroke include loss of function of the affected region and death.

Pulmonary embolism results from the migration of the embolus from a formation site within the deep veins of the extremities into the pulmonary vasculature. In the event of an acute blockage, consequences include rapid death by heart failure. Pulmonary hypertension frequently results.

Formation of thrombi within the deep veins of the lower extremities is characterized as deep vein thrombosis. Causative factors include blood stasis. Certain surgical procedures also correlate strongly with postoperative venous clot formation. These include hip or knee replacement, elective neurosurgery, and acute spinal cord injury repair.

Therapeutic lysis of pathogenic thrombi is achieved by administering thrombolytic agents. Benefits of thrombolytic therapy include rapid lysis of the thromboembolic disorder and restoration of normal circulatory function. Complications include internal and external bleeding due to lysis of physiologic clots, and stroke, resulting in cerebral hemorrhage. Currently available treatments include administration of streptokinase, anistreplase, urokinase, or tissue plasminogen activator (TPA).

The efficacy of thrombolytic therapy in the treatment of myocardial infarction has been demonstrated over the past ten years using one or more of the agents described above. Unfortunately, there are side effects associated with these agents. For example, TPA is associated with secondary toxicity, such as hypofibrinogenemia and bleeding. Also, successful application of thrombolytics in ischemic stroke has not been realized.

### SUMMARY OF THE INVENTION

It is an object of the present invention to overcome the aforesaid deficiencies of the prior art.

It is another object of the present invention to provide agents that induce platelet fragmentation and lysis.

It is a further object of the present invention to provide agents that dissolve platelet arterial thrombi generally found in the coronary arteries of patients with acute myocardial infarction as well as other arterial occlusions.

It is another object of the present invention to provide agents that generate hydrogen peroxide in the vicinity of platelets so that the platelets are fragmented.

It has been discovered that anti-platelet GPIIIa49-66 Ab induces platelet oxidation, fragmentation and death by activating platelet 12-lipoxygenase, generating 12(S)-HETE (hydroxy eicosatetraenoic acid) and NADPH-oxidase with exposure of membrane fragment phosphatidyl serine and thrombin-generating capacity.

An IgG antibody has been found which induces thrombocytopenia and platelet fragmentation and correlates with thrombocytopenia in patients with HIV-1-related thrombocytopenia. This antibody reacts with platelet epitope GPIIIa49-66 on platelet membranes. The mechanism of platelet fragmentation is induced by hydrogen peroxide generated by the antibody. The present inventors have discovered that platelets contain the NADPH oxidase pathway, which is used by granulocytes to kill bacteria.

This antibody, or a monoclonal antibody derived from the GPIIIa49-66 epitope, will dissolve arterial thrombi generally found in the coronary arteries of patients with acute myocardial infarction, as well as other arterial occlusions. The F(ab′)₂ fragment of this antibody generates the same number of platelet fragments as intact IgG, but induces considerably less murine thrombocytopenia, ˜40% of the efficacy of the intact IgG.

A monoclonal anti-GPIIIa 49-66 antibody can be engineered to have the same “homing site” as tissue plasminogen activator for fibrin. Fibrin is interspersed within the arterial thrombus. The N-terminal part of the TPA molecule contains five kringles between amino acids 83-550 which contain the lysine binding sites for substrate proteins. The second kringle has a binding site specific for fibrin. This fusion protein can be used to dissolve platelet thrombi, either alone or in combination with TPA, A23187, or PMA.

Yet another agent for inducing oxidation of platelets is A23187, also known as calcium ionophore A23187 or calcimycin.

Additionally, PMA (phorbol 12-myristate 13-acetate), a PKC (protein kinase C) activator, has been found to induce oxidation of platelets.

Recombinant ADAMTS-18, a disintegrin and metalloprotease with thrombospondin-like motifs induced platelet oxidation/fragmentation in an identical kinetic fashion as anti-GPIIIa49-66 Ab.

These agents can be used alone, in combination with each other, or with conventional agents such as TPA, urokinase, and streptokinase.

### BRIEF DESCRIPTION OF THE DRAWINGS

FIG. 1 shows box plot comparisons of PEG-IC protein concentration, size and sGPIIb antigen in control subjects and HIV-1-ITP patients. Mean is shown by the solid black box; median by the horizontal line in the large open box; 25^{th} and 75^{th} percentiles by the lower and upper border of the large open box from which spread of the data from the position of the median can be assessed. Whiskers include 99% of a Gaussian distribution. A. Protein concentration, n=22 controls and 46 HIV-1-ITP patients. B. Size determined by forward light scatter, n=22 controls and 46 HIV-1-ITP patients. C. GPIIb determined by MoAb-FITC, n=15 controls and 35 HIV-1-ITP patients.

FIG. 2 is a flow cytometry histogram of platelet particle formation. Three panels represent: CTL, buffer alone; CTL IgG, IgG isolated from control PEG-IC; PT IgG, IgG isolated from HIV-1-ITP patient PEG-IC. Numbers in left upper quadrant refer to % particles in that quadrant.

FIG. 3 shows the effect of time, concentration and temperature on platelet particle formation. FIG. 3A, Time dependent platelet particle formation induced by control (o-o) vs patient (l-l) PEG-IC IgG. FIG. 3B, Concentration dependence of control (open column) vs patient (dark column) platelet particle formation. FIG. 3C, Temperature dependence of control vs patient platelet particle formation.

FIG. 4 shows distribution of % platelet particle formation in control vs HIV-1-ITP vs ATP Patients. IgG from 12 control, 16 HIV-1-ITP patients and 5 ATP patients is given.

FIG. 5 is a comparison of rabbit vs patient anti-GPIIIa 49-66 induced platelet particle formation. Preimmune rabbit and patient control IgG reactivity are cited under CTL.

FIG. 6 illustrates platelet particle formation induced by control and patient IgG, F(ab′)2 and Fab fragments, at 40, 28 and 56 ug/ml respectively for 4 hrs at 37° C., n=6. SEM is given. Difference between patient F(ab′)2 vs Fab is significant at the p<0.05 level, student t test.

FIG. 7 shows the effect of Anti-GPIIIa49-66 Ab on platelet count and platelet particle formation in control and complement deficient mice. C57BL/6 control and C3−/− deficient mice were injected i.p. with 25 ug of control ( ) or anti-GPIIIa49-66 Ab and platelet count monitored at various time intervals.

FIG. 8 shows the effect of anti-GPIIIa 49-66 on platelet particles in vitro. The percentage of platelet particles was monitored at 4 hrs. WT refers to wild type; Mut to C3−/− mice, n=5 for each group, SEM is given. FIG. 8 illustrates the effect on anti-GPIIIa 49-66 on platelet particle fragmentation in vivo.

FIG. 9 shows the effect of anti-GPIIIa 49-66 IgG vs. F(ab′)₂ on platelet count.

FIG. 10A is an in vivo comparison of intact anti-GPIIIa49-66 IgG vs its F(ab′)₂ Fragment on platelet count and platelet particle formation. FIG. 10A shows the effect of IgG or F(ab′)₂ on platelet particles formation. In FIG. 10A, Balb/c mice were injected i.p. with 25 ug of patient IgG or 17 ug of control F(ab′)₂ or patient F(ab′)₂ and platelet count monitored at various time intervals. FIG. 10B shows the effect of peroxide inhibitors, catalase and diphenylenidonium on platelet particle formation induced by anti-GPIIIa49-66 Ab at 4 Hrs. c refers to control IgG, ci to control IgG plus inhibitor at highest concentration employed. Bars after 50 uM/ml catalase and 5 uM/ml DPI refer to doubling concentrations. n=4, SEM is given.

FIG. 11 shows electron microscopy of damaged platelets treated with anti-GPIIIa49-64 antibodies. FIG. 11 A shows a patient sample at one hour showing platelets with a fuzzy material attached to the outer surface of the cell membranes (dotted arrows). Gaps are noted in the cell membranes with leakage of cytoplasmic content (arrows). These areas are shown at higher magnification in FIGS. 11 B and C. None of these changes was present in IgG controls at four hours, as shown in FIG. 11 D. The original magnifications for FIGS. A and D were 4000; for 11B, 50,000; for 11C, 40,000.

### DETAILED DESCRIPTION OF THE INVENTION

Immunologic thrombocytopenia is a common complication of HIV-1 infection [1-3]. Kinetic studies on platelet survival strongly suggest that early-onset HIV-1-ITP is secondary to increased peripheral destruction of platelets, whereas patients with AIDS are more likely to have decreased platelet production [4]. Patients with early-onset HIV-1-ITP have a thrombocytopenic disorder that is indistinguishable from classic autoimmune thrombocytopenia (ATP), seen predominantly in females [1, 5-8]. However, HIV-1-ITP is different from classic ATP with respect to male predominance and markedly elevated platelet-associated IgG, IgM, complement protein C3 and C4, as well as the presence of circulating serum immune complexes (CIC's) composed of the same [6, 7]. Past studies have revealed that these complexes contain anti-platelet integrin GPIIIa (b3) Ab [9], and its anti-idiotype blocking Ab [10], as well as other Ab's and their anti-idiotypes [11-13].

Affinity purification of anti-platelet GPIIIa Ab from CIC's of these patients has revealed a high affinity IgG1 [9] reactive against a specific sequence within the GPIIIa protein corresponding to residues 49-66 [10]. The presence of anti-GPIIIa49-66 Ab correlates inversely with platelet count (r=0.71) and induces severe thrombocytopenia in mice [10] (mouse GPIIIa is 83% homologous with human GPIIIa, and macrophages have Fc receptors for human IgG1). Murine thrombocytopenia can be prevented or reversed with GPIIIa49-66 peptide [10], as well as anti-idiotype blocking Ab [14].

CIC anti-GPIIIa49-66 Ab can be removed by centrifugation [10]. This suggested the presence of particulate platelet membrane fragments within the CIC. The presence of these fragments in HIV-1-ITP serum has been documented by demonstrating the presence of platelet membrane receptor antigen GPIIIa as well as GPIIb and GPIb in the CIC's of these patients, and it has been shown that platelet fragments can be induced in vitro and in vivo with anti-GPIIIa49-66 Ab. It has also been found that Ab-mediated fragmentation is complement-independent and occurs via a novel mechanism involving the generation of hydrogen peroxide by stimulation of an NADPH oxidase pathway in platelets.

It has been discovered that anti-platelet GPIIIa49-66 Ab induces platelet oxidation, fragmentation and death by activating platelet 12-lipoxygenase, generating 12(S)-HETE (hydroxy eicosatetraenoic acid) and NADPH-oxidase.

Recent studies by the present inventors have demonstrated that activation of oxidative platelet death requires classic Ca⁺⁺ flux (fura-2, AM), which is completely inhibited by 100 μM EGTA (ethylenebis(oxyethylenenitrilo)tetraacetic acid) or 10 microM BAPTA (1,2-bis (o-aminophenoxy)ethane-N.N.N′,N′-tetraacetic acid), a calcium-specific chelator. Cell oxidative platelet death is associated with mild GPIIIa activation. Oxidative fragmentation/death is not induced in the presence of 1 μM PGE1, 10 μM dibutyl cyclic AMP (n=6) and occurs in Gaq KO mouse platelets (all conditions which inhibit ADP, collagen or thrombin-induced platelet activation).

It has also been discovered that platelet oxidation/fragmentation can be induced independently of anti-GPIIIa49-66 by 10 mM A23187, a Ca⁺⁺ ionophore, or 0.4 μM PMA, a protein kinase C activator. Both A23187 and PMA induce oxidation of platelets loaded with the oxidative fluorochrome, DCHF (dichlorofluorescein). Their reactivity is inhibited by the oxidation scavengers catalase (H₂O₂) and DPI (diphenylene iodonium, an inhibitor of NADPH-oxidase), and is absent in NADPH oxidase p47phox(−/−), gp91phox(−/−)KO as well as 12-LO(−/−)KO mouse platelets. Thus, anti-GPIIIa49-66 could be inducing the intracellular effects of ionophore and PMA.

To discover a possible physiologic mechanism, platelet GPIIIa49-66 was panned with a phage-peptide display library. Twenty 7-mer peptide clones were found which reacted with GPIIIA49-66. One of these peptides, VHCVQLY, had 70% homology with ADAMTS-18, a disintegrin and metalloprotease with thrombospondin (TSP)-like motifs, constitutively secreted by endothelial cells. An 18-mer peptide of ADAMTS-18 was then synthesized from the C-terminal TSP motif and conjugated to biotin, bio-VQTRSVHCVQQGRPSSSC-OH. The peptide alone had no effect on platelet oxidation/fragmentation. However, an anti-biotin antibody used to cluster the peptide did induce oxidation/fragmentation (n=6).

Recombinant ADAMTS-18 was then made with the expression vector pBudCE4.1 in 293T cells. This peptide induced platelet 12(S)-HETE and oxidation/fragmentation in an identical kinetic fashion as anti-GPIIIa49-66 antibody. Both expressed rADAMTS-18, and HUVEC conditioned media ADAMTS-18 could be activated by thrombin (0.5μ/mL and then neutralized with hirudin), with optimum effect at one hour (n=4). HUVEC ADAMTS-18 induced oxidation fragmentation could be inhibited about 50% by an scFV antibody raised against the ADAMTS-18 (18-mer) peptide as well as GPIIIa49-66 peptide, as well as RGDS (GPIIIa ligand binding site) (n=7).

Both peptides GPIIIa49-66 and RGDS were synergistic (˜75% inhibited) when combined at optimum individualized concentration, suggesting that there are two binding sites on platelet GPIIIa.

Thus, a mechanism is proposed for platelet thrombus clearance, induced by platelet membrane oxidative fragmentation leading to thrombin generation and activation of constitutively secreted endothelial cell ADAMTS-18.

### Material and Methods

Human Population. Patient sera were obtained from 46 early-onset HIV-1-infected patients without AIDS: 12 control subjects (healthy laboratory personnel) and 5 classic ATP patients.

Mouse Population. Female BALB/c, B6129 and C57BL/6 mice were obtained from Taconic Farms. C3(−/−) mice, C57BL/6 were kindly provided by Dr. Harvey Colton, Northwestern University Medical School, Chicago, Ill. NADPH deficient mice (p47phox(phagocyte oxidase) (−/−)) were kindly provided by Dr. Harry L. Malech, NIAID, Bethesda, Md.

F(ab′)₂ and Fab. F(ab′)₂ fragments were prepared from purified IgG by pepsin digestion as described [15], and were shown to be free of Fc fragments by SDS-PAGE as well as ELISA [15], Fab fragments were prepared by papain digestion of IgG as described [15] and verified by SDS-PAGE.

Immune Complexes. Circulating immune complexes (CIC's) were isolated from serum by polyethylene glycol precipitation (PEG-IC) [6, 14]. Precipitates were dissolved in one fifth their serum volume in 0.01M PBS, pH 7.4.

Isolation of IgG and IgM from Immune Complexes. IgG and IgM were isolated and purified as described [9]. In brief, polyethylene glycol (PEG)-ICs were applied to a staphylococcal protein A affinity column (Sigma-Aldrich). The bound complex was washed with PBS and eluted with 0.1M glycine buffer, pH 2.5. The eluted material was applied to an acidified Sephadex G-200 gel filtration column (Amersham Pharmacia Biotech) preequilibrated with the same elution buffer. Effluents of the IgG peak were isolated, neutralized, dialyzed against PBS, and applied to a rabbit anti-IgM affinity column (ICN Pharmaceuticals, Inc.) prepared from Affi-Gel 10 (BioRad). The flow-through material was free of contaminating IgM by immunoblot and ELISA. Effluents of the IgM peak were isolated, neutralized, dialyzed against PBS, and applied to an anti-Fc receptor affinity column to remove rheumatoid factor. Fc fragments were prepared by papain digestion [15] and affinity purified on a staphylococcal protein A column; the acid eluate was verified by SDS-PAGE and was coupled to Affi-Gel 10. The flow-through IgM was devoid of rheumatoid factor, as determined by inability to bind to a second Fc column.

Affinity Purification of Anti-Platelet IgG. Antiplatelet IgG was affinity purified with 10⁸ platelets fixed with 2% paraformaldehyde for 2 hr at room temperature, followed by overnight gentle rocking at 4° C., then acid elution and neutralization, as described [9]. The IgG subclass, determined by radial immunodiffusion (The Binding Site), was IgG1 with both k and l light chains.

Affinity Purification of Anti-Platelet GPIIIa49-66. Peptide GPIIIa49-66, CAPESIEFPVSEARVLED (synthesized by Quality Controlled Biochemicals), was coupled to an affinity column with the heterobifunctional cross-linker sulfo-succinimidyl 4-(N-maleimidomethyl)cyclohexane-1-carboxylate as recommended by the manufacturer (Pierce Chemical Co.; cross-links the resin with NH₂-terminal cysteine of the peptide), and was incubated with 0.4 ml of affinity-purified IgG overnight at 4° C. The column was then washed, eluted at pH 2.5, and neutralized as described [9].

Induction of Platelet Particles. Gel-filtered platelets were prepared from blood collected in 0.38% sodium citrate employing a Sepharose 2B column preincubated with Tyrode's buffer. 1×10⁷ gel-filtered platelets/ml were labelled with an anti-GPIIb-FITC monoclonal Ab (MoAb) (3B2) [16] or an anti-GPIIIa-FITC MoAb (Ancell, Bayport, Minn.), 10 ug/ml for 30 min at 4° C., centrifuged at 1000 g×6 min at room temperature, and resuspended in Tyrodes buffer. 10 μl of FITC-labelled platelets (10⁷/ml) were then incubated with 15 μl of affinity-purified anti-GPIIIa49-66 (20-80 ug/ml) and 75 μl Tyrodes buffer for 0-4 hrs at 37° C. and then stored in an ice bucket prior to measurement of % platelet particles by flow cytometry. Further particle formation is arrested at 0° C. (see below).

For mouse in vivo studies, blood was collected from orbital sinus or by cardiac puncture into a heparinized syringe after anesthesizing mice with metafane (Schering-Plough Animal Health, Union, N.J.). Platelet-rich plasma was prepared and incubated with MoAb anti-mouse CD41 (Integrin aIIb chain, Pharmingen, San Diego, Calif.) for 30 min at 4° C. and then assayed directly by flow cytometry.

Assay of Platelet Particle Formation. % platelet particles were measured by flow cytometry, employing an Epics Elite Cell Sorter (Coulter, Hialeah, Fla.). Debris and dead cells were excluded using scatter gates. Only cells with low orthogonal light scattering were included in the sorting gates. Gates were adjusted for control platelets by exclusion of other blood cells. Intact platelets were monitored in the right upper quadrant (RUQ) with the Y-axis measuring forward-scatter and the X-axis measuring fluorescence. A shift in the fluorescent particles from RUQ to LUQ reflected % platelet particle induction of 10,000 counted platelets/particles.

ELISA Assays. CIC GPIIIa and phosphatidylserine was measured by ELISA. GPIIIa was measured by incubating 25 ug of PEG-IC with 10 ug/ml MoAb 3B2-FITC in 0.1M final volume for 30 min at 4° C., and then assayed by flow cytometry.

Preparation of Rabbit Anti-GPIIIa 49-66. GPIIIa49-66 was synthesized by Quality Control Biochemicals (Hopkinton, Mass.). Antibody was prepared commercially by Cocalico Biologicals, Inc (Reamstown, Pa.) employing KLH-conjugated GPIIIa49-66 with 4 booster injections 21-77 days post primary injection of 500 ug.

Electron Microscopy. Platelets were suspended in agar and fixed in 3% glutaraldehyde in 0.1-M sodium cacodylate buffer. Samples were washed twice in buffer, post-fixed with 1.5% osmium tetroxide and rewashed 2× with buffer. Samples were then dehydrated and embedded in Eponate-12 resin. Thin sections were cut in a Reichert Ultracut 5 ultramicrotome, counterstained with uranyl acetate and lead citrate, and analyzed using a Zeiss EM-10 electron microscope.

Materials: All reagents were obtained from Sigma (St. Louis, Mo.) unless otherwise designated. PDC980598 (MAPKinase inhibitor) was obtained from Research Biochemicals Inc., Natick, Mass. Anti-caspases 1 and 3 and BAPTA-AM were obtained from Molecular Probes, Eugene, Oreg. MoAb's against platelet GPIIIa (LK6-55, LK7r, LK3r, LK4-r5, and CG4 were produced in our laboratory [18]). MoAb against GPIba (1b10) was a gift from Dr. Zaverio Ruggeri, Scripps Research Institute (La Jolla, Calif.).

### Results

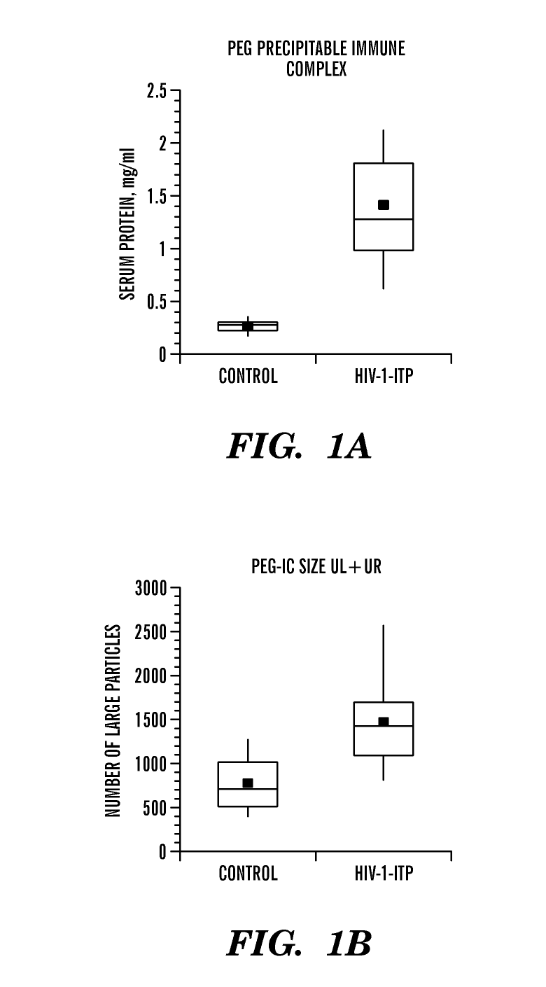

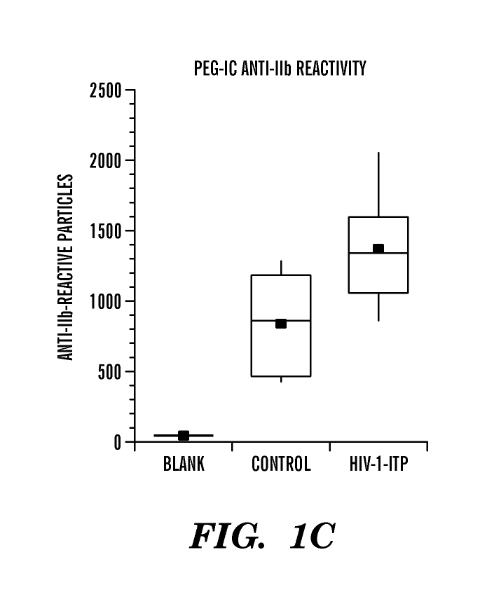

Detection of Platelet Glycoproteins in PEG-IC's of HIV-1-ITP Patients. Previous results have shown increased serum concentration of CIC in patients with HIV-1-ITP and that these CIC's contain Ab specific for GPIIIa49-66. We confirmed and extended these results in the population studied. FIG. 1A demonstrates a 5.5 fold greater protein concentration of PEG-IC's derived from 46 HIV-1-ITP patients compared to 22 normal control subjects.

PEG-IC size was also measured in a similar cohort of patients. FIG. 1B demonstrates a 2 fold greater size in 35 HIV-1-ITP patients compared to 15 control subjects as determined by forward light scatter.

In a previous report, the loss of ˜75% of anti-GPIIIa49-66 activity in PEG-IC following centrifugation at 100,000 g for 1 hr suggested the presence of platelet membrane fragments in the IC's [10]. This was confirmed by immunoblot of the IC's with MoAb's vs GPIIIa and GPIba (data not shown). This observation was more extensively investigated by an analysis of IC samples from 35 patients with HIV-1-ITP compared to 15 control subjects (FIG. 1C). This revealed 1.7 fold greater platelet GPIIb than control subjects (P=0.005, Student t test). The GPIIb found in control IC preparations is due to the expected presence of platelet fragments in serum.

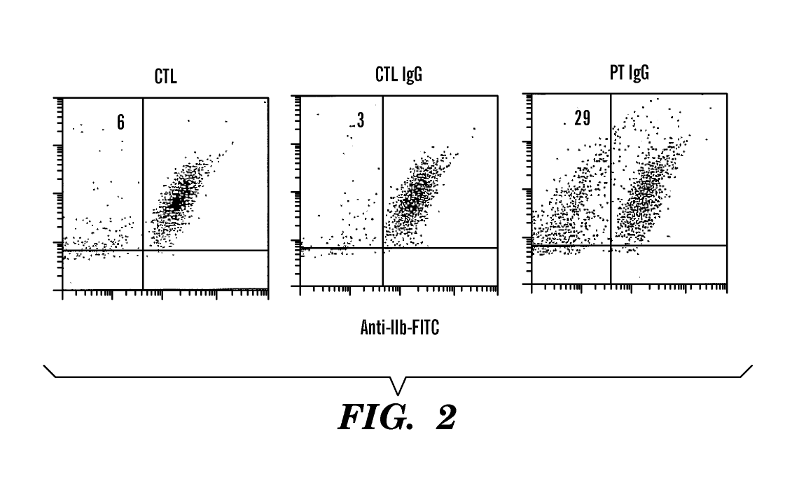

Antibody Specific for GPIIIa49-66 Induces Platelet Fragmentation In Vitro. The presence of platelet membrane antigens in PEG-IC's of HIV-1-ITP patients suggested that anti-GPIIIa49-66 Ab could be inducing these changes. To investigate this possibility, gel-filtered platelets were incubated with affinity-purified anti-GPIIIa49-66 Ab in the absence of serum or complement. FIG. 2 shows the flow cytometric analysis of 1 such experiment in which anti-GPIIb-FITC-labelled platelets shifted their fluorescence intensity and distribution from the RUQ to the lower end of the LUQ, indicating platelet fragmentation.

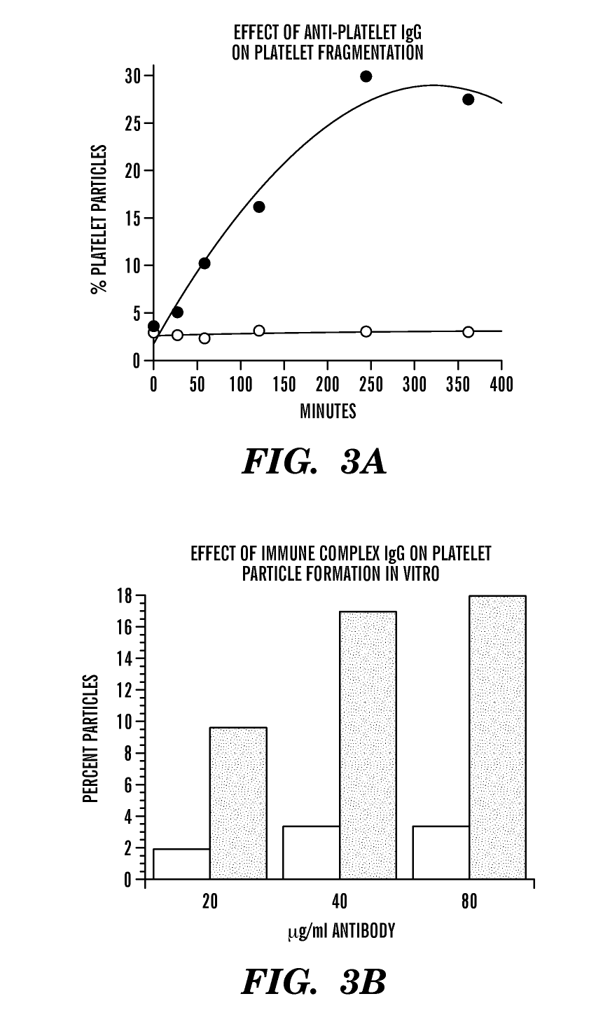

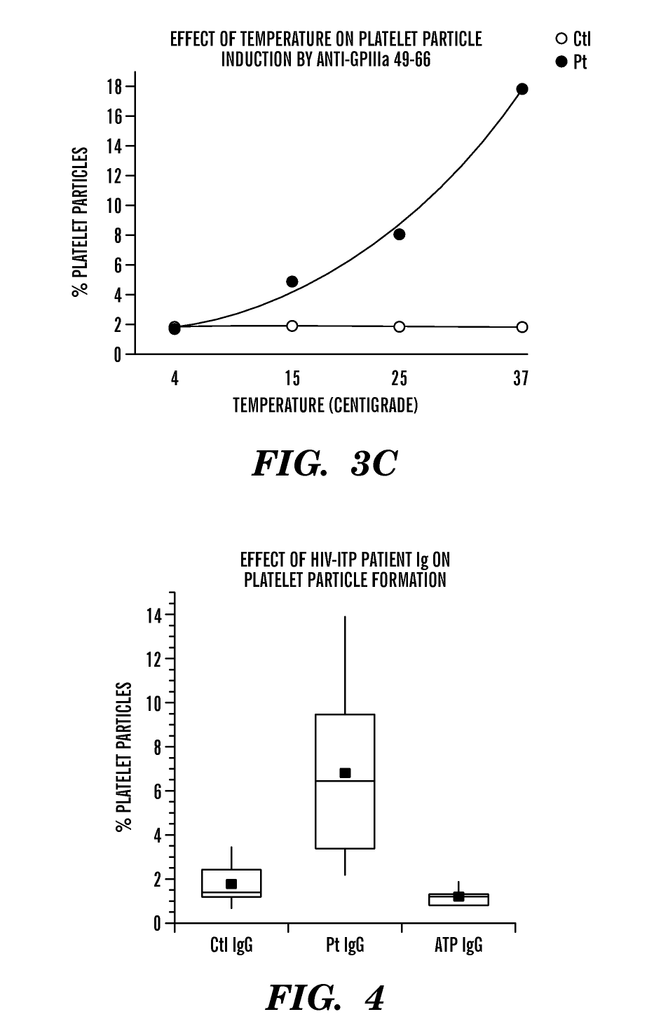

Analysis of Time, Concentration and Temperature Dependence of Platelet Fragmentation Induced by Anti-GPIIIa49-66. FIG. 3A demonstrates optimum platelet particle formation at 4 hrs, employing 25 μg/ml anti-GPIIIa 49-66 Ab. This represents ˜30% of enumerated events.

FIG. 3B shows concentration-dependence of platelet particle formation, with optimum concentration at 40 ug/ml.

FIG. 3C demonstrates temperature dependence of platelet particle formation. Inactivity at 4° C., permitted overnight storage of samples prior to analysis by flow cytometry, whenever necessary.

Induction of Platelet Fragmentation in HIV-1-ITP vs Classic ATP Patients. FIG. 4 demonstrates the platelet particle formation distribution in 16 HIV-1-ITP patients compared to 5 ATP patients and 12 control subjects. Note the ˜5 fold greater platelet particle formation in HIV-1-ITP patients compared to control subjects or ATP patients.

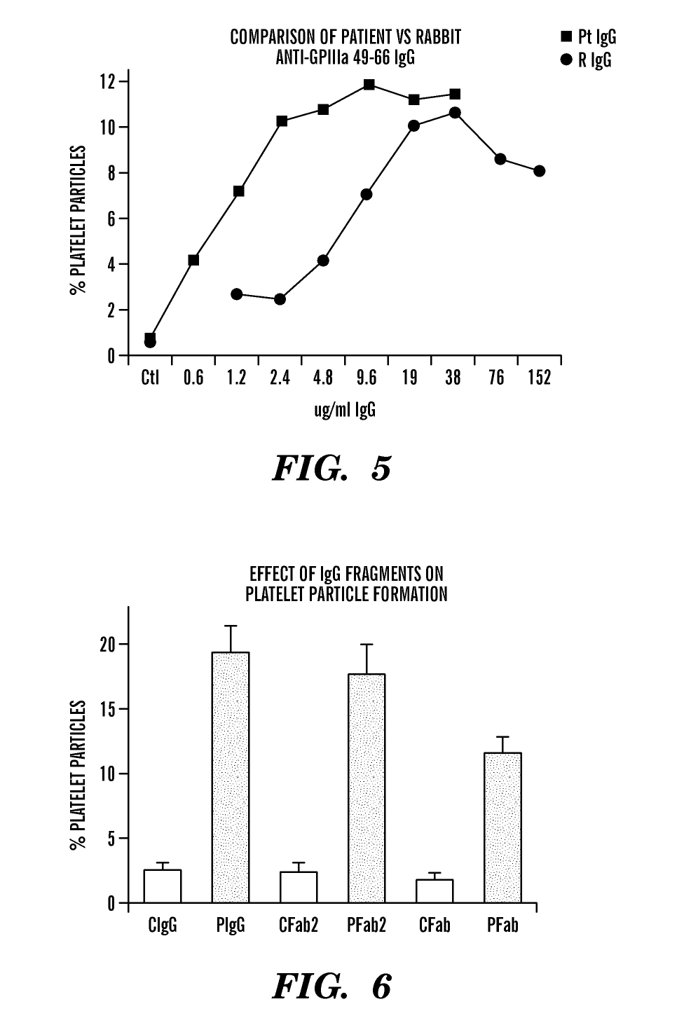

Specificity of Anti-GPIIIa49-66 for Platelet Fragmentation. Table 1 demonstrates the inability of 6 different anti-GPIIIa MoAb's with different specificities for GPIIIa [18], as well as 1 anti-GPIba MoAb to induce platelet particle formation. To confirm this striking result, an anti-GPIIIa49-66 Ab were raised in rabbits, affinity-purified it against fixed platelets and then reacted it with gel-filtered platelets. FIG. 5 demonstrates the similar property of platelet particle formation compared to non-immune rabbit IgG, albeit at an 8 fold lower avidity.

TABLE 1
Specificity of Ab-Induced Platelet Particle Formation
% of Platelet Particles

| | Zero Time | 2 Hrs | 4 hrs |
|---|---|---|---|
| PEG-IC IgG | | | |
| CTL | 0.80 | 0.55 | 0.50 |
| PT | 0.87 | 11.1 | 19.7 |
| MoAb Anti-GPIIIa | | | |
| LK6-55 | 0.83 | 0.76 | 0.50 |
| CG4 | 0.81 | 0.55 | 0.81 |
| LK7r | 0.75 | 0.54 | 0.63 |
| LK3r | 0.75 | 0.53 | 1.20 |
| LK5-50 | 0.59 | 0.56 | 0.94 |
| LK4-55 | 0.91 | 0.70 | 0.62 |
| MoAb Anti-GPIbα | | | |
| anti-Ib | 0.69 | 0.68 | 0.71 |

Gel-filtered platelets were incubated with various IgG antibodies from control PEG-IC, patient PEG-IC, murine MoAbs against GPIIIa and a MoAb against GPIbα and 0, 2 and 4 hrs and then assayed for platelet particle formation.

Platelet Fragmentation Induced by F(ab′)₂ and Fab Fragments. FIG. 6 demonstrates platelet particle formation with F(ab′)₂ fragments indicating that complement was unlikely to be involved in this reaction. Of interest is the positive result obtained with 2 fold molar equivalent Fab fragments albeit at ˜60% the effective platelet particle formation of F(ab′)₂ fragments (p<0.05, Student t test), suggesting the possibility that dimerization of GPIIIa, could play a role.

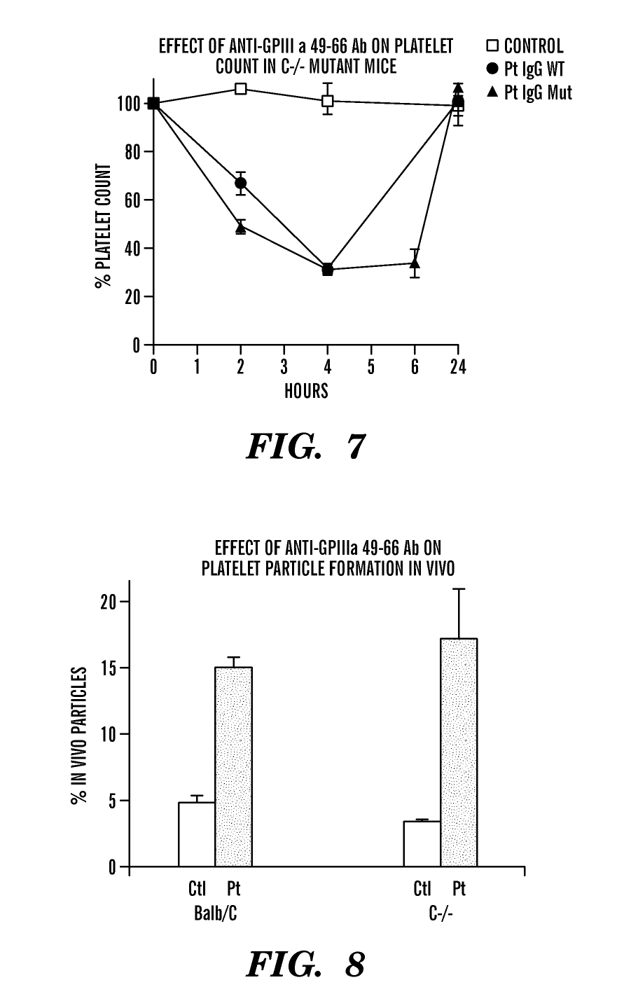

Induction of Thrombocytopenia and Platelet Fragmentation in Complement Deficient C3(−/−) Mice. The ability to generate platelet particles in vitro, in the absence of the Fc domain of anti-platelet GPIIIa49-66 strongly suggested that platelet particle formation was independent of complement fixation. Nevertheless, complement deposition on cell membranes can induce membrane vesiculation (as well as cell lysis), and it is possible that complement may play a role in platelet fragmentation in vivo. We therefore attempted to induce thrombocytopenia in complement-deficient, C3(−/−) as well as wild-type mice. FIGS. 7A and B document similar thrombocytopenia induction and platelet particle formation in both wild-type and C3(−/−) mice, indicating that complement is not required for platelet fragmentation and thrombocytopenia.

Induction of Thrombocytopenia and Platelet Fragmentation with F(ab′)₂ Fragments. Induction of thrombocytopenia in complement deficient mice indicated that in vivo thrombocytopenia was not due to complement-mediated cell clearance, but likely to be due to clearance of opsonized platelets as well as platelet fragmentation. The role of these two mechanisms was analyzed by measuring the contribution of F(ab′)₂ fragments vs intact IgG. FIG. 8A indicates that thrombocytopenia could be induced in the absence of the Fc domain of IgG but at 40% the efficiency of intact IgG. Similarly platelet particle formation could also be induced in vivo at 75% the efficacy of intact IgG, FIG. 8B. Thus, clearance of opsonized platelets and fragments can take place in the absence of Ab binding to Fc receptors on phagocytic cells; perhaps by other phagocytic scavenger mechanisms ( ).

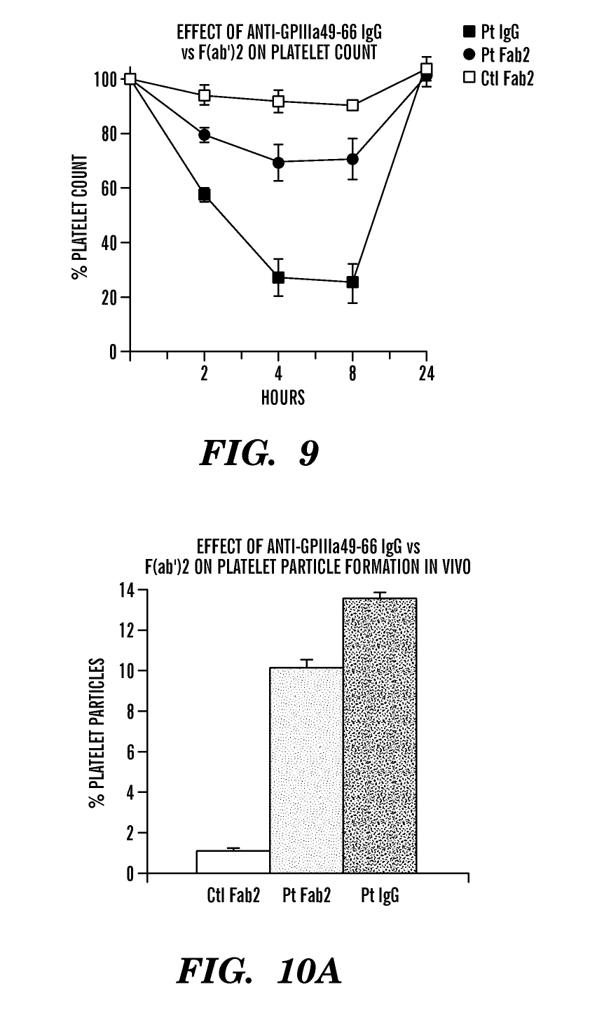

Induction of Platelet Fragmentation via Anti-GPIIIa49-66 is Implemented by the Generation of Peroxide. Numerous attempts to elucidate the mechanism(s) of Ab-induced platelet particle formation were unsuccessful. These included inhibitors of anaerobic and aerobic glycolysis (3 mM 2-deoxyglucose, 10 mM NaA_{z}), microtubules (2 mM Colchicine, 0.2 mM vinblastine), microfilaments (10 uM cytochalasin D), calpain (100 uM calpastatin, 5 ug/ml leupeptin), apoptosis (100 uM general caspase inhibitor FK-011 and caspases 1 and 3), protease inhibitors (5 ug/ml leupeptin, 2 mM PMSF, 5 uM SBTI, 5000 u/ml aprotonin) and various intracellular signalling kinases: 2 uM Wortmannin (PI3Kinase), 200 uM staurosporine (phospholipid/Ca⁺⁺ dependent protein kinase), 40 uM H-7 (serine/threonine kinase), and 200 uM PDC980598 (MAPKinase). However, a recent report by Lerner and coworkers [19] provided evidence that Ab's in general are capable of inducing peroxide formation from an assortment of Ag's depending upon the tryptophan and cysteine composition and orientation. This reaction required the generation of singlet ¹0₂ via irradiation with UV or visible light. This is followed by Ab-Ag induced reduction of ¹0₂ to 0⁻₂ (superoxide) with consequent generation of H₂0₂ which could be neutralized with catalase. We therefore studied the effect of catalase on platelet particle formation and noted that it could inhibit the reaction in the absence of UV/light irradiation (FIG. 9). Three other oxidase inhibitors failed to inhibit Ab-mediated platelet particle formation: 20 uM indomethacin against cyclooxygenase, 200 uM allopurinol against xanthine oxidase and 200 uM L-N-monomethylarginine against NO synthetase (data not shown). This suggested that ¹0₂ could be generated by another mechanism, such as a cellular generating system such as the NADH/NADPH oxidase system. This hypothesis was tested with the use of diphenyleneiodonium (DPI), an inhibitor of NADH/NADPH oxidase, as well as other flavoprotein oxidases. FIG. 9 demonstrates inhibition by DPI in a similar manner as catalase.

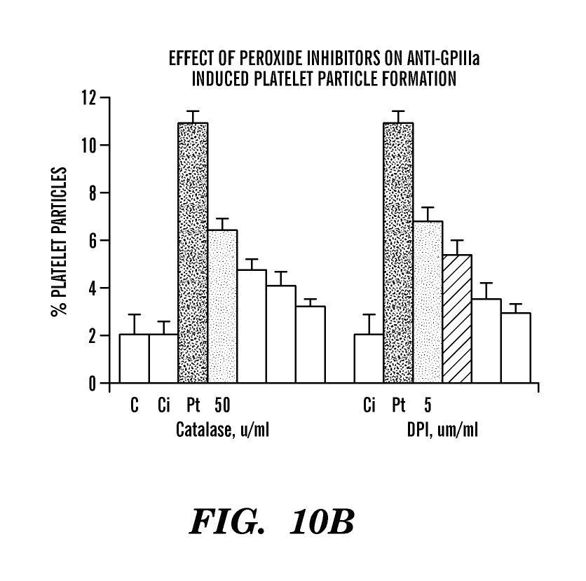

Induction of Thrombocytopenia and Platelet Fragmentation in NADPH-Deficient (P47phox(−/−)) Mice. Inhibition of platelet fragmentation by inhibitors of H₂O₂ generation suggested that platelets contain a peroxide generating pathway, namely the NADPH oxidase system present in granulocytes/phagocytes [20]. In vivo experiments were therefore performed in p47phox(−/−) mice deficient in the p47 component of the phagocytic oxidase complex necessary for H₂0₂ generation via the NADPH oxidase pathway. FIG. 10A demonstrates that thrombocytopenia induced in p47 phox (−/−) mice by anti-GPIIIa49-66 Ab was ˜40% of that obtained with wild type C57/BL mice, with no difference noted between F(ab′)₂ fragment and IgG preparations. FIG. 10B demonstrates absence of platelet particle formation in p47phox(−/−) mice, compared to 13% platelet particle formation in wild type mice. These data indicate that platelet particle formation is induced by H₂0₂ damage generated by Ab-induced activation of the NADPH oxidase pathway and that platelet fragmentation contributes to platelet clearance.

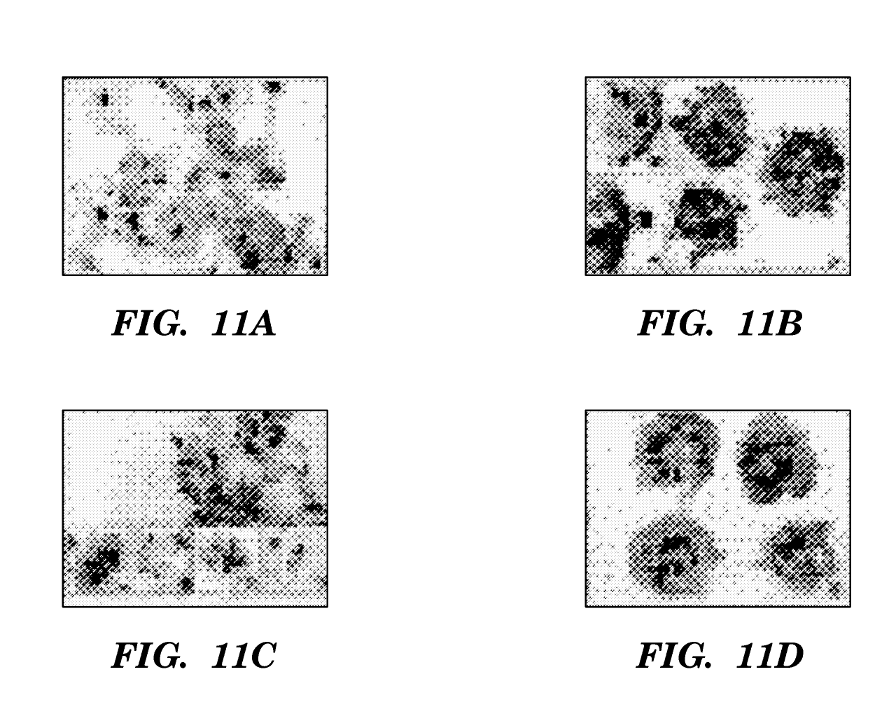

Electron Microscopy of Platelet Fragmentation Induced by Anti-GPIIIa49-66 Ab. FIG. 11 demonstrates the dramatic progressive platelet damage induced by anti-GPIIIa49-66 antibody at 1 and 4 hrs of incubation. Ab-damaged platelets develop breaks in their membrane, swelling and release of cytoplasmic fragments. At 1 hr platelets had cytoplasmic-like material attached to the external surface of their membranes (FIG. 11A,B,C). Cytoplasmic contents leaked out of the platelet through gaps in the membranes and adhered to the outer surface but the granules are preserved (FIG. 11B,C). Some platelets show vacuolization. At 4 hrs most platelets showed signs of cellular injury. Many were swollen and others showed partial or almost total disintegration of their cell membrane. Dense and other granules were unaffected. Clumping of cellular debris with platelet fragments was also seen. No such changes were noted with control IgG-treated platelets. The supernatant collected from the centrifuged platelet samples consisted mostly of cell debris with occasional degenerating platelets (data not shown). Of interest is the observation that a minority of platelets (perhaps young platelets [21]) appear resistant to this Ab damage.

### Discussion

These data reveal a new pathophysiologic mechanism for platelet destruction (fragmentation) involving peroxide damage generated by an NADPH oxidase pathway in platelets. This peroxide can be generated by an autoantibody specific for a platelet GPIIIA49-66 epitope, which is complement-independent. Complement independence is documented by Ab-induced microparticle formation with F(ab′)₂ fragments in vitro, and Ab-induced thrombocytopenia and microparticle formation in C3 (−/−) mice in vivo. Peroxide damage was documented by inhibition of Ab-induced platelet fragmentation by peroxide inhibitors, catalase and DPI in vitro, and inhibition of microparticle formation and thrombocytopenia in p47phox (−/−) and gp91phox (−/−) mice.

The anti-platelet GPIIIa49-66 antibody induces platelet oxidation, fragmentation and death by activating platelet 12-lipoxygenase, generating 12(S)-HETE and NADPH-oxidase. This activation of oxidative platelet death requires Ca2⁺⁺ flux.

Platelet oxidation/fragmentation can be induced independently of anti-GPIIIa49-66 by A23187, a Ca2⁺⁺ iononphore, by phorbol 12-myristate 13-acetate (PMA), and recombinant ADAMTS-18.

Membrane shedding or “microparticle formation” is a normal property of cells grown in culture [22-24], as well as cells undergoing apoptosis [25, 26]. Platelet microparticle formation is enhanced by numerous pathophysiologic conditions relating to platelet activity, such as agonist-induced platelet activation with thrombin, collagen or Ca ionophore A1237 [27-29]; complement-induced platelet lysis [30]; immunologic destruction of platelets in autoimmune thrombocytopenia [31-33] and heparin-induced thrombocytopenia [34, 35]; shear stress in cardiopulmonary bypass [36-39], severe arterial stenosis [40]; and other thrombocytic conditions such as thrombotic thrombocytopenia [41], disseminated intravascular coagulation [17, 42], and transient ischemic attacks [43].

Platelet microparticles induced by platelet agonists have been reported to contain GPIIb/GPIIIa, GPIb [29, 30], CD9 [44], P-selectin [30, 36, 44] and Factor V [30] and to require Ca⁺⁺ [45] calpain [28, 45-47], caspase 3 [27] and intact GPIIb/GPIIIa [48] for their formation. Whether platelet microparticles with potential bioactive properties contribute to the pathophysiology of disease or are a secondary consequence has not been resolved.

The ability of an Ab to induce platelet fragmentation by reactivity with a specific epitope on platelet membrane GPIIIa via elaboration of platelet generated peroxide is unique. The sequence specificity of anti-GPIIIa Ab in inducing platelet fragmentation by the peroxide-dependent mechanism is supported by our finding that 5 other anti-GPIIIa MAb's against at least 4 different regions of GPIIIa [18] as well as a MoAb against GPIb to induce a similar reaction are ineffective. This intriguing observation was confirmed using a rabbit Ab raised against GPIIIa49-66 which gave a similar platelet fragmentation histogram, albeit at 8 fold less avidity, with preimmune rabbit IgG having no effect. These observations suggest the possibility of a conformational change induced at a specific region of GPIIIa which is capable of activating a peroxide-generating pathway in platelets.

Peroxide-induced platelet membrane damage is supported by several observations: platelet microparticle formation is: 1) inhibited by catalase, a peroxide scavenger, 2) inhibited by DPI, an inhibitor of flavoprotein oxidases, not by inhibitors of other oxidases: cyclooxygenase, xanthine oxidase, NO synthetase 3) inhibited by superoxide dismutase, 4) absent in p47phox(−/−) and gp91phox(−/−) mice which are incapable of generating peroxide by this pathway. The absence of platelet particle formation and attenuation of thrombocytopenia in p47phox(−/−) mice indicates that platelets contain the NADPH oxidase complex pathway and that this is the pathway utilized for peroxide generation in mouse platelets.

The present observations on platelet destruction and microparticle formation with IgG as well as F(ab′)₂ fragments, in both wildtype and C3(−/−) mice, as well as abrogation of this effect in p47phox(−/−) mice strongly indicate that platelet destruction can be via a platelet fragmentation mechanism induced by peroxide generation with clearance by other than classic Fc or complement receptors.

The antibodies and peptides of the present invention include functional derivatives of these antibodies and peptides. By “functional derivative” is meant a fragment, variant, analog, or chemical derivative of the subject antibody or peptide, which terms are defined below. A functional derivative retains at least a portion of the amino acid sequence of the antibody or peptide of interest, which permits its utility in accordance with the present invention, namely, induction of platelet fragmentation. This specificity can readily be quantified by means of the techniques described above.

A “fragment” of the antibodies and peptides disclosed herein refers to any subset of the molecule, that is, a shorter peptide. Fragments of interest, of course, are those which induce a high degree of platelet fragmentation.

A “variant” of the antibodies or peptides refers to a molecule which is substantially similar either to the entire antibody or peptide a fragment thereof. Variant peptides may be conveniently prepared by direct chemical synthesis of the variant peptide, using methods well known in the art.

Alternatively, amino acid sequence variants of the antibodies and peptides of the present invention can be prepared by mutations in the DNAs which encode the antibody or peptide of interest. Such variants include, for example, deletions from, or insertions or substitutions of, residues within the amino acid sequence. Any combination of deletion, insertion, and substitution may also be made to arrive at the final construct, provided that the final construct possesses the desired activity. Obviously, the mutations that will be made in the DNA encoding the variant peptide must not alter the reading frame, and preferably will not create complementary regions that could produce secondary mRNA structure.

At the genetic level, these variants ordinarily are prepared by site-directed mutagenesis of nucleotides in the DNA encoding the antibody or peptide molecule, thereby producing DNA encoding the variant, and thereafter expressing the DNA in recombinant cell culture. The variants typically exhibit the same qualitative biological activity as the nonvariant antibody, i.e., they fragment platelets.

An “analog” of the antibodies or peptides disclosed herein refers to a non-natural molecule which is substantially similar to either the entire antibody or peptide or to an active fragment thereof.

A “chemical derivative” of an antibody or peptide contains additional chemical moieties which are not normally part of the amino acid sequence of the antibody. Covalent modifications of the amino acid sequence are included within the scope of this invention. Such modifications may be introduced into the antibody or peptide derivatives by reacting targeted amino acid residues from the peptide with an organic derivatizing agent that is capable of reacting with selected side chains or terminal residues.

The types of substitutions which may be made in the antibodies or peptides herein may be based on analysis of the frequencies of amino acid changes between a homologous protein of different species. Based upon such analysis, conservative substitutions may be defined herein as exchanges within one of the following five groups:
I. Small, aliphatic nonpolar or slightly polar residues:
   Ala, Ser, Thr, Pro, Gly
II. Polar, negatively charged residues and their amides:
   Asp, Asn, Glu, Gln
III. Polar, positively charged residues:
   His, Arg, Lys
IV. Large, aliphatic nonpolar residues:
   Met, Leu, Ile, Val, Cys
V. Large aromatic residues
   Phe, Tyr, Trp

Within the foregoing groups, the following substitutions are considered to be “highly conservative”:

Asp/Glu

His/Arg/Lys

Phe/Tyr/Trp

Met/Leu/Val

Pharmaceutical compositions for administration can comprise at least one antibody or peptide or fragment derivative or variant thereof as disclosed herein in a pharmaceutically acceptable form, optionally combined with a pharmaceutically acceptable carrier, and/or further optionally combined with another clot-dissolving agent such as streptokinase, urokinase or TPA. These compositions can be administered by any means that achieve their intended purposes. Amounts and regimens for the administration of a composition according to the present invention can be determined readily by those with ordinary skill in the art of treating thromboembolic disorders, including ischemic stroke, myocardial infarction, or pulmonary embolism.

Compositions of the present invention can be administered in the same way as TPA, and can be administered alone or in combination with TPA, etc. For example, administration can be by parenteral, such as subcutaneous, intravenous, intramuscular, intraperitoneal, transdermal, or buccal routes. The dosage administered depends upon the age, health and weight of the recipient, type of previous or concurrent treatment, if any, frequency of the treatment, and the nature of the effect desired.

Compositions within the scope of this invention include at least all compositions comprising at least one antibody or peptide disclosed herein, fragments, derivatives, or fragments thereof in an amount effective to achieve its intended purpose. While individual needs vary, determination of optimal ranges of effective amounts of each component is within the skill of the art. Typical dosages comprise about 0.1 to about 10 mg/kg body weight for humans (25 μg/20 gm mouse).

It should also be understood that to be useful, the treatment provided need not be absolute, provided that it is sufficient to carry clinical value. An agent which provides treatment to a lesser degree than do competitive agents may still be of value if the other agents are ineffective for a particular individual, if it can be used in combination with other agents to enhance the overall level of protection, or if it is safer than competitive agents.

It is understood that the suitable dose of a composition according to the present invention will depend upon the age, sex, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

Prior to use in humans, a drug is first evaluated for safety and efficacy in laboratory animals. In human clinical trials, one begins with a dose expected to be safe for humans, based on the preclinical data for the drug in question, and on customary doses for analogous drugs, if any. If this dose is effective, the dosage may be increased to determine the minimum effective dose, if desired. If this dose is ineffective, the dosage may be decreased to determine the minimum effective dose, if desired. If this dose is ineffective, it will be cautiously increased, with the patients monitored for signs of side effects. See, e.g., Berkow et al., eds., The Merck Manual, 15^{th} edition, Merck and Co., Rahway, N.J., 1987; Goodman et al., eds, Goodman and Gilmans The Pharmacological Basis of Therapeutics, 8^{th} edition, Pergamon Press, Inc., Elmsford, N.Y. (1990); Avery's Drug Treatment: Principles and Practice of Clinical Pharmacology and Therapeutics, 3^{rd} edition, ADIS Press, LTD., Williams and Wilkins, Baltimore, Md. (1987); Ebadi, Pharmacology, Little, Brown and Co., Boston (1985), which references and references cited therein are entirely incorporated herein by reference.

The total dose required for each treatment may be administered in multiple doses or in a single dose. The compositions may be administered alone or in conjunction with other therapeutics directed to the disease or directed to other symptoms thereof.

In addition to the compounds disclosed herein, a pharmaceutical composition may contain suitable pharmaceutically acceptable carriers, such as excipients, carriers and/or auxiliaries which facilitate processing of the active compounds into preparations which can be used pharmaceutically.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Abrams, C. S., Ellison, N., Budzynski, A. Z., and Shattil, S. J., “Direct detection of activated platelets and platelet-derived microparticles in humans”, Blood 75:128-138 (1990)
Armstrong, M. J., Storch, J., and Dainiak, N., “Stimulating distinct plasma membrane regions gives rise to extracellular membrane vesicles in normal and transformed lymphocytes”, Biochim. Biophys. Acta. 946:106-112 (1988)
Aupeix, K., Hugel, B., Martin, T., Bischoff, P., Lill, H., Pasquali, J.-L., and Freyssinet, J. M., “The significance of shed membrane particles during programmed cell death in vitro, and in vivo, in HIV-1 infection”, J. Clin. Invest. 99:1546-1554 (1997)
Barry, O. P., Pratico, D., Savani, R. C., and FitzGerald, A. “Modulation of monocyte-endothelial cell interactions by platelet microparticles”, J. Clin. Invest. 102:136-144 (1998)
Beaudoin, A. R., and Grondin, G. Shedding of vesicular material from the cell surface of eukaryocytic cells: different cellular phenomena”, Biochim. Biophys. Acta. 1071:203-219 (1991)
Fox, J. E. B., Austin, C. D., Reynolds, C. C., and Steffen, P. K., “Evidence that agonist-induced activation of calpain causes the shedding of procoagulant-containing microvesicles from the membrane of aggregating platelets”, J. Biol. Chem. 266:13289-13295 (1991)
Gemmell, C. H., Sefton, M. V., and Yeo, E. L., “Platelet-derived microparticle formation involves glycoprotein IIb-IIIa Inhibition by RGDS and a Glanzmann's thrombasthenia defect”, J. Biol. Chem. 268:14586-14589 (1993)
George, J. N., Pickett, E. B., Saucerman, S., McEver, R. P., Kunicki, T. J., Kieffer, N., and Newman, P. J., “Platelet surface glycoproteins. Studies on resting and activated platelets and platelet membrane microparticles in normal subjects, and observations in patients during adult respiratory distress syndrome and cardiac surgery”, J. Clin. Invest. 78:340-348 (1986)
Holme, P. A., Muller, F., Solum, N. O., Brosstad, F., Froland, S. S., and Aukrust, P., “Enhanced activation of platelets with abnormal release of RANTES in human immunodeficiency virus type 1 infection”, FASEB 12:79-89 (1998)
Holme, P. A., Orrim, M. J., Hamers, M. J. A. G., Solum, N. O., Brosstad, F. R., Barstad, R. M., and Sakariassen, K. S., “Shear-induced platelet activation and platelet microparticle formation at blood flow conditions as in arteries with a severe stenosis”, Arteriosclerosis 17:646-653 (1997)
Holme, P. A., Solum, N. O., Brosstad, F., Pedersen, T., and Kveine, M., “Microvesicles bind soluble fibrinogen, adhere to immobilized fibrinogen, and coaggregate with platelets”, Thromb. Haemost 79:389-394 (1998)
Holme, P. A., Solum, N. O., Brosstad, F., Roger, M., and Abdelnoor, M. “Demonstration of platelet-derived microvesicles in blood from patients with activated coagulation and fibrinolysis using a filtration technique and Western blotting”, Thrombosis Haemost. 72:666-671, (1994)
Hughes, M., Hayward, C. P. M., Warkentin, T. E., Horsewood, P., Chorneyko, K. A., and Kelton, J. G., “Morphological analysis of microparticle generation in heparin-induced thrombocytopenia”, Blood 96:188-194 (2000)
Inngjerdingen, M., Waterhouse, K., and Solum, N. O., “Studies on the dual effects on platelets of a monoclonal antibody to CD9, and on the properties of platelet CD9”, Thrombosis Res. 95:215-227 (1999)
J., and Niewiarowski, S. Measurement of platelet microparticles during cardiopulmonary bypass by means of captured ELISA for GPIIb/IIIa, Thrombosis Haemost. 80:225-230 (1998)
Jansen, P. G. M., Have, K. T., Eijsman, L., Hack, C. E., and Sturk, A., “Cell-derived microparticles generated in patients during cardiopulmonary bypass are highly procoagulant”, Circulation 96:3534-3541 (1997).
Jost, J., Tauber, M. G., Luthy, R., and Siegenthaler, W., “HIV-assozierte Thrombozytopenie”, Schweiz Med Wschr. 118:206-212 (1988)
Jy, W., Horsman, L. L., Arce, M., and Ahn, Y. S., “Platelet microparticles in ITP”, J Lab Clin Med. 119, 119-334 (1992)
Jy, W., Horstman, L. L., Arce, M., and Ahn, Y. S. (1992). Clinical significance of platelet microparticles in autoimmune thrombocytopenias. J. Lab. Clin. Med. 119, 334-345.
Karpatkin, S. (1969). Heterogeneity of human platelets. I. Biochemical and kinetic evidence suggestive of young and old platelets. J Clin Invest. 29, 1073-1083.
Karpatkin, S. (1997). Autoimmune (idiopathic) thrombocytopenic purpura. Lancet. 349, 1531-1536.
Karpatkin, S., Nardi, M. A., and Hymes, K. B. (1995). Sequestration of anti-platelet GPIIIa antibody in Rheumatoid Factor-immune complexes of human immunodeficiency virus 1 thrombocytopenic patients. Proceedings of the National Academy of Sciences U.S.A. 92, 2263-2267.
Karpatkin, S., Nardi, M. A., and Kouri, Y. (1992). Internal image anti-idiotype HIV-1gp120 antibody in human immunodeficiency virus 1 (HIV-1)-seropositive individuals with thrombocytopenia. Proc Natl Acad Sci USA. 89, 1487-1491.
Karpatkin, S., Nardi, M. A., Lennette, E. T., Byrne, B., and Poiesz, B. (1988). Anti-human immunodeficiency virus type 1 antibody complexes on platelets of seropositive thrombocytopenic homosexuals and narcotic addicts. Proc Natl Acad Sci USA. 85, 9763-9767.
Karpatkin, S., Xia, J., Patel, J., and Thorbecke, G. (1992). Serum platelet-reactive IgG of ATP patients is not F(ab′)2 mediated and a function of storage. Blood. 80, 3164-3172.
Kelton, J. G., Moore, J. C., Warkentin, T. E., and Hayward, C. P. M. (1996). Isolation and characterization of cysteine proteinase in thrombotic thrombocytopenic purpura. Brit. J. Haematol. 93, 421-426.
Khan, I., Zucker-Franklin, D., and Karpatkin, S. (1975). Microthrombocytosis and platelet fragmentation associated with idiopathic/autoimmune thrombocytopenic purpura. Brit. J. Haemat. 31, 449-460.
Leaf, A. N., Raphael, B., Hochster, H., Laubenstein, L. J., Baez, L., and Karpatkin, S., “Thrombotic thrombocytopenic purpura associated with HIV-1 infection”, Ann Intl Med. 109:194-197 (1988)
Lee, Y. J., Jy, W., Horstman, L. L., Janania, J., Reyes, Y., Kelley, R. E., and Ahn, Y. S. (1994). Elevated platelet microparticles in transient ischemic attacks, lacunar infarcts, and multiinfarct dementias. Thromb. Res. 72, 295-304.
Liu, L.-X., Nardi, M. A., Nierodzik, M. L., and Karpatkin, S. (1995). Heterogeneous inhibition of platelet aggregation by MoAb's binding to multiple sites on GPIIIa. Brit J. Haematol. 91, 976-982.
Mallat, Z., and et al (1999). Shed membrane microparticles with procoagulant potential in human atherosclerotic plaques: a role for apoptosis in plaque thrombogenicity. Circulation. 99, 348-353.
Miyamoto, S., Marcinkiewicz, C., Edmunds, L. H. Morris, L., Distenfeld, A., Amorosi, E., and Karpatkin, S. (1982). Autoimmune thrombocytopenic purpura in homosexual men. Ann Int Med. 96, 714-717.
Murphy, M. F., Metcalfe, P., and Waters, A. H. (1987). Incidence and mechanism of neutropenia and thrombocytopenia in patients with human immunodeficiency virus infection. Brit J Haematol. 66, 337-340.
Najean, Y., and Rain, J.-D. (1994). The mechanism of thrombocytopenia in patients with HIV. J Lab Clin Med. 123, 415.
Nardi, M. A., Liu, L.-X., and Karpatkin, S. (1997). GPIIIa (49-66) is a major pathophysiologically-relevant antigenic determinant for anti-platelet GPIIIa of HIV-1-related immunologic thrombocytopenia (HIV-1-ITP). Proceedings of the National Academy of Sciences U.S.A. 94, 7589-7594.
Nardi, M., and Karpatkin, S. (2000). Antiidiotype antibody against platelet anti-GPIIIa contributes to the regulation of thrombocytopenia in HIV-1-ITP patients. J Exper Med. 191, 2093-2100.
Nieuwland, R. N., Berckmans, R. J., McGregor, S., Boing, A. N., Romijn, F. P. H. T. M., Westendroop, R. G. J., Hack, C. E., and Sturk, A. (2000). Cellular origin and procoagulant properties of microparticles in meningococcal sepsis. Blood. 95, 930-935.
Nomura, S., Suzuki, M., Katsura, K., Xie, G. L., Miyazaki, Y., Miyake, T., Kido, H., Kagawa, H., and Fukuhara, S. (1995). Platelet-derived microparticles may influence the development of atherosclerosis in diabetes mellitus. Atherosclerosis. 116, 235-240.
Owens, M. R., Holme, S., and Cardinali, S. (1992). Platelet microvesicles adhere to subendothelium and promote adhesion of platelets. Thromb. Res. 66, 247-258.
Ratnoff, O. D., Menitove, J. E., Aster, R. H., and Lederman, M. M. (1983). Coincident classic hemophilia and “idiopathic” thrombocytopenic purpura in patients under treatment with concentrates of anti-hemophilic factor (factor VIII). N Engl J Med. 308, 439-442.
Savona, S., Nardi, M. A., and Karpatkin, S. (1985). Thrombocytopenic purpura in narcotics addicts. Annals of Internal Medicine. 102, 737-741.
Segal, B. H., Leto, T. L., Gallin, J. I., Malech, H. L., and Holland, S. M. (2000). Genetic, Biochemical and Clinical Features of Chronic Granulomatous Disease. Medicine. 79, 170-200.
Segundo, C., Medina, F., Rodriguez, C., Martinez-Palencia, R., Leyva-Cobian, F., and Brieva, J. A. (1999). Surface molecule loss and bleb formation by human germinal center B cells undergoing apoptosis: role of apoptotic blebs in monocyte chemotaxis. Blood. 94, 1012-1020.
Shcherbina, A., and Remold-O'Donnell, E. (1999). Role of caspase in a subset of human platelet activation responses. Blood. 93, 4222-4231.
Sims, P. J., Faioni, E. M., Weidmer, T., and Shattil, S. J. (1988). Complement proteins C5b-9 cause release of membrane vesicles from the platelet surface that are enriched in the membrane receptor for coagulation factor Va and express prothrombinase activity. J Biol Chem. 263, 18205-18212.
Sims, P. J., Wiedmer, T., Esmon, C. T., Weis, H. J., and Shattil, S. J. (1989). Assembly of the platelet prothrombinase complex is linked to vesiculation of the platelet membrane. Studies in Scott syndrome: an isolated defect in platelet procoagulant activity. J Biol Chem. 264, 17049-17057.
Varon, D., and Karpatkin, S. (1983). A monoclonal anti-platelet antibody with decreased reactivity for autoimmune thrombocytopenic platelets. Proc Natl Acad Sci USA. 80, 6992-6995.
Walsh, C. M., Nardi, M. A., and Karpatkin, S. (1984). On the mechanism of thrombocytopenic purpura in sexually-active homosexual men. New England Journal of Medicine. 311, 635-639.
Warkentin, E. T., Hayward, C. P. M., Boshkov, L. K., and et al (1994). Sera from patients with heparin-induced thrombocytopenia generate platelet-derived microparticles with procoagulant activity: an explanation for the thrombotic complications of heparin-induced thrombocytopenia. Blood. 84, 3691-3699.
Weidmer, T., Shattil, S. J., Cunningham, M., and Sims, P. J. (1990). Role of calcium and calpain in complement-induced vesiculation of the platelet plasma membrane and in the exposure of the platelet factor Va receptor. Biochemistry. 29, 623-632.
Wentworth, A. D., Jones, L. H., Wentworth, P. J., Janda, K. D., and Lerner, R. A. (2000). Antibodies have intrinsic capacity to destroy antigens. Proc Natl Acad Sci. 97, 10930-10935.
Wolf, B. B., Goldstein, J. C., Stennicke, H. R., Beere, H., Amarante-Mendes, G. P., Salvesen, G. S., and Green, D. R. (1999). Calpain functions in a caspase-independent manner to promote apoptosis-like events during platelet activation. Blood. 94, 1683-1692.
Yano, Y., Shiba, E., Kambyashi, J.-I., and et al (1993). The effects of calpeptin (a calpain specific inhibitor) on agonist induced microparticle formation from the platelet plasma membrane. Thromb. Res. 71, 385-396.
Yu, J.-R., Lennette, E. T., and Karpatkin, S. (1986). Anti-F(ab′)2 antibodies in thrombocytopenic patients at risk for Acquired Immunodeficiency Syndrome. J Clin Invest. 77, 1756-1761.
Zucker-Franklin, D., and Karpatkin, S. (1977). Erythrocyte and platelet fragmentation in idiopathic autoimmune thrombocytopenic purpura. New Engl J Med. 297, 517-523.

What is claimed is:

1. A method for inducing platelet fragmentation in a subject, said method comprising:
selecting a subject with a condition requiring platelet fragmentation and
administering to the selected subject an antibody raised against the protein (GPIIIa (49-66) or a binding portion thereof, which recognizes the protein (GPIIIa (49-66), under conditions effective to induce platelet fragmentation.

2. The method of claim 1, wherein an antibody raised against the protein (GPIIIa (49-66) is administered.

3. The method of claim 1, wherein a monoclonal antibody is administered.

4. The method of claim 1, wherein a binding portion of an antibody raised against the protein GPIIIa (49-66) is administered.

5. The method of claim 1, wherein said administering is carried out parenterally, intravenously, intramuscularly, intraperitoneally, transdermally, or buccally.

6. A method for treating thromboembolic disorders in a subject, said method comprising:
selecting a subject with a thromboembolic disorder and
administering to the selected subject an effective amount of an antibody raised against the protein GPIIIa (49-66) or a binding portion thereof, which recognizes the protein GPIIIa (49-66), under conditions effective to treat the thromboembolic disorder.

7. The method according to claim 6, wherein the thromboembolic disorder is selected from the group consisting of ischemic heart disease, ischemic stroke, deep vein thrombosis, and pulmonary embolism.

8. The method of claim 6, wherein an antibody raised against the protein (GPIIIa (49-66) is administered.

9. The method of claim 6, wherein a monoclonal antibody is administered.

10. The method of claim 6, wherein a binding portion of an antibody raised against the protein GPIIIa (49-66) is administered.

11. The method of claim 6, wherein said administering is carried out parenterally, intravenously, intramuscularly, intraperitoneally, transdermally, or buccally.
